(19) United States
(12) Patent Application Publication
(10) Pub. No.: US 2024/0317944 A1
(43) Pub. Date: Sep. 26, 2024

(54) POLYURETHANE-BASED RADIOPAQUE CROSSLINKED HYDROGELS WITH NON-ISOCYANATE PRECURSOR FOR MEDICAL APPLICATIONS

(71) Applicant: Boston Scientific Scimed, Inc., Maple Grove, MN (US)

(72) Inventor: Yen-Hao Hsu, Shrewsbury, MA (US)

(73) Assignee: Boston Scientific Scimed, Inc., Maple Grove, MN (US)

(21) Appl. No.: 18/615,619

(22) Filed: Mar. 25, 2024

Related U.S. Application Data
(60) Provisional application No. 63/492,071, filed on Mar. 24, 2023.

(51) Int. Cl. C08J 3/075; C08G 18/10; C08G 18/30; C08J 3/24

(52) U.S. Cl. CPC C08J 3/075; C08G 18/10; C08G 18/30; C08J 3/24; C08J 2375/04

(57) ABSTRACT

In some aspects, a system for forming a hydrogel composition is described that comprises (a) a multifunctional cyclic carbonate compound and (b) a radiopaque, reactive polymer that comprises a plurality of amino groups that are reactive with the multifunctional cyclic carbonate groups of the multifunctional cyclic carbonate compound. In other aspects, a radiopaque crosslinked hydrogel composition is described that comprises a crosslinked reaction product of a multifunctional cyclic carbonate compound and a radiopaque, reactive polymer that comprises a plurality of amino groups. In further aspects, a method of treatment is described that comprises administering to a subject a mixture that comprises a multifunctional cyclic carbonate compound and a radiopaque, reactive polymer that comprises a plurality of amino groups, wherein the mixture is administered under conditions such that the multifunctional cyclic carbonate compound and the radiopaque, reactive multi-arm polymer crosslink after administration.

### CROSS-REFERENCE TO RELATED APPLICATIONS

This application claims the benefit of U.S. Provisional Patent Application Ser. No. 63/492,071 filed on Mar. 24, 2023, the disclosure of which is incorporated herein by reference.

### FIELD

The present disclosure relates radiopaque hydrogels and to crosslinkable systems for forming radiopaque hydrogels, among other aspects. The radiopaque hydrogels and crosslinkable systems for forming the same are useful, for example, in various medical applications.

### BACKGROUND

Bioresorbable hydrogels with rapid crosslinking reaction rate in vivo, known by the trade name of SpaceOAR®, have become a prominent biomaterial and obtained clinical success in creating the space between prostate and rectum, tremendously improving patient safety during the cancer therapies. A further improvement based on this application is that some of 8-Arm PEG branches are functionalized with 2,3,5-triiodobenzamide (TIB) groups, replacing part of the activated ester end groups, succinimidyl glutarate (SG), in order to provide intrinsic radiopacity to the hydrogels themselves for CT-visibility. This hydrogel, known by the trade name of SpaceOAR Vue®, is the next generation of SpaceOAR® for prostate medical applications.

While the above approach is effectual, the hydrophobic TIB group has solubility limitation in water that creates trade-offs with regard to how many of TIB groups can be functionalized while maintaining ideal hydrophilic properties for the PEG. Moreover, the amount of TIB functionalization plays a key role with regard to tuning the radiopacity to satisfy physicians' needs; however, sacrificing ester end groups purposes of introducing TIB groups results in poor or non-ideal crosslinking density, which can increase swelling after injection which also reducing gel radiopacity and persistence.

For these and other reasons, alternative strategies for forming iodine-labelled crosslinked hydrogels that provide enhanced crosslink density per star polymer are desired.

### SUMMARY

The present disclosure provides an alternative approach to that described above.

In some aspects, the present disclosure is directed to a system for forming a hydrogel composition that comprises (a) a multifunctional cyclic carbonate compound and (b) a radiopaque, reactive polymer that comprises a plurality of amino groups that are reactive with the multifunctional cyclic carbonate groups of the multifunctional cyclic carbonate compound.

In some embodiments, which can be used in conjunction with the above aspects, the multifunctional cyclic carbonate compound comprises two or more cyclic carbonate groups selected from five-membered cyclic carbonate groups, six-membered cyclic carbonate groups, seven-membered cyclic carbonate groups and eight-membered cyclic carbonate groups.

In some embodiments, which can be used in conjunction with the above aspects and embodiments, the radiopaque, reactive polymer comprises a plurality of hydrophilic polymer arms comprising end moieties that comprise one or more radiopaque halogen groups and one or more reactive amino groups. The hydrophilic polymer arms may comprise, for example, one or more hydrophilic monomers selected from ethylene oxide, propylene oxide, N-vinyl pyrrolidone, oxazoline monomers, hydroxyethyl acrylate, hydroxyethyl methacrylate, PEG methyl ether acrylate or PEG methyl ether methacrylate, or PNIPAAM. The end moieties may comprise, for example, a monocyclic or multicyclic aromatic structure that is substituted with (a) one or more iodine groups and (b) one or more amino groups or amino-containing groups.

In some embodiments, which can be used in conjunction with the above aspects and embodiments, the end moieties are linked to the hydrophilic polymer arms through a hydrolysable ester group.

In some embodiments, which can be used in conjunction with the above aspects and embodiments, the system further comprises one or more additional agents selected from therapeutic agents, imaging agents, colorants, tonicity adjusting agents, suspension agents, wetting agents, and pH adjusting agents.

In some embodiments, which can be used in conjunction with the above aspects and embodiments, the system comprises (a) a first composition that comprises the multifunctional cyclic carbonate compound and the radiopaque, reactive polymer and (b) an accelerant composition.

In some embodiments, which can be used in conjunction with the above aspects and embodiments, the system comprises a delivery device.

In some aspects, the present disclosure is directed to a radiopaque crosslinked hydrogel composition comprising a crosslinked reaction product of (a) a multifunctional cyclic carbonate compound and (b) a radiopaque, reactive polymer that comprises a plurality of amino groups that are reactive with the multifunctional cyclic carbonate groups of the multifunctional cyclic carbonate compound.

In some embodiments, which can be used in conjunction with the above aspects, the radiopaque crosslinked hydrogel composition further comprises one or more additional agents selected from therapeutic agents, imaging agents, colorants, tonicity adjusting agents, suspension agents, wetting agents, and pH adjusting agents.

In some embodiments, which can be used in conjunction with the above aspects and embodiments, the multifunctional cyclic carbonate compound comprises two or more cyclic carbonate groups selected from five-membered cyclic carbonate groups, six-membered cyclic carbonate groups, seven-membered cyclic carbonate groups and eight-membered cyclic carbonate groups.

In some embodiments, which can be used in conjunction with the above aspects and embodiments, the radiopaque, reactive polymer comprises a plurality of hydrophilic polymer arms comprising end moieties that comprise one or more radiopaque halogen groups and one or more reactive amino groups. The hydrophilic polymer arms may comprise, for example, one or more hydrophilic monomers selected from ethylene oxide, propylene oxide, N-vinyl pyrrolidone, oxazoline monomers, hydroxyethyl acrylate, hydroxyethyl methacrylate, PEG methyl ether acrylate or PEG methyl ether methacrylate, or PNIPAAM. The end moieties may comprise, for example, a monocyclic or multicyclic aromatic structure that is substituted with (a) one or more iodine groups and (b) one or more amino groups or amino-containing groups.

In some embodiments, which can be used in conjunction with the above aspects and embodiments, the end moieties are linked to the hydrophilic polymer arms through a hydrolysable ester group.

In some aspects, the present disclosure pertains to a method of treatment comprising administering to a subject a mixture that comprises (a) a multifunctional cyclic carbonate compound and (b) a radiopaque, reactive polymer that comprises a plurality of amino groups that are reactive with the multifunctional cyclic carbonate groups of the multifunctional cyclic carbonate compound, wherein the mixture is administered under conditions such that the multifunctional cyclic carbonate compound and the radiopaque, reactive multi-arm polymer crosslink after administration.

In some embodiments, which can be used in conjunction with the above aspects, the method of treatment comprises administering to the subject a fluid composition that has a basic pH and comprises the mixture of the multifunctional cyclic carbonate compound and the radiopaque, reactive polymer, and wherein the fluid composition crosslinks after administration in vivo to form a radiopaque crosslinked hydrogel composition.

In some embodiments, which can be used in conjunction with the above aspects and embodiments, the method of treatment comprises administering (a) a first fluid composition that has an acidic pH and comprises mixture of the multifunctional cyclic carbonate compound and the radiopaque, reactive polymer and (b) a second fluid composition that is buffered to a basic pH, wherein the first and second fluid compositions combine to form a fluid composition that comprises the mixture of the multifunctional cyclic carbonate compound and the radiopaque, reactive polymer and has a basic pH, and wherein the fluid composition crosslinks in vivo to form a radiopaque crosslinked hydrogel composition.

In some embodiments, which can be used in conjunction with the above aspects and embodiments, the mixture of the multifunctional cyclic carbonate compound and the radiopaque, reactive polymer further comprises one or more additional agents selected from therapeutic agents, imaging agents, colorants, tonicity adjusting agents, suspension agents, wetting agents, and pH adjusting agents.

In some embodiments, which can be used in conjunction with the above aspects and embodiments, the multifunctional cyclic carbonate compound comprises two or more cyclic carbonate groups selected from five-membered cyclic carbonate groups, six-membered cyclic carbonate groups, seven-membered cyclic carbonate groups and eight-membered cyclic carbonate groups.

In some embodiments, which can be used in conjunction with the above aspects and embodiments, the radiopaque, reactive polymer comprises a plurality of hydrophilic polymer arms comprising end moieties that comprise one or more radiopaque halogen groups and one or more reactive amino groups. The hydrophilic polymer arms may comprise, for example, one or more hydrophilic monomers selected from ethylene oxide, propylene oxide, N-vinyl pyrrolidone, oxazoline monomers, hydroxyethyl acrylate, hydroxyethyl methacrylate, PEG methyl ether acrylate or PEG methyl ether methacrylate, or PNIPAAM. The end moieties may comprise, for example, a monocyclic or multicyclic aromatic structure that is substituted with (a) one or more iodine groups and (b) one or more amino groups or amino-containing groups.

In some embodiments, which can be used in conjunction with the above aspects and embodiments, the end moieties are linked to the hydrophilic polymer arms through a hydrolysable ester group.

Potential benefits associated with the present disclosure include one or more of the following: radiocontrast is maintained, crosslink density is enhanced, and in vivo persistence is obtained.

The above and other aspects, embodiments, features and benefits of the present disclosure will be readily apparent from the following detailed description.

### BRIEF DESCRIPTION OF THE DRAWINGS

FIG. 1 schematically illustrates a method of forming a radiopaque, reactive polymer, in accordance with an embodiment of the present disclosure.

FIG. 2 schematically illustrates a method of forming a polyurethane-based radiopaque crosslinked hydrogel, in accordance with an embodiment of the present disclosure.

FIG. 3 illustrates a delivery device, in accordance with an embodiment of the present disclosure.

FIG. 4 illustrates a delivery device, in accordance with another embodiment of the present disclosure.

### DETAILED DESCRIPTION

In several aspects of the present disclosure, a polyurethane-based radiopaque crosslinked hydrogel is provided that comprises a crosslinked reaction product of (a) a multifunctional cyclic carbonate compound and (b) a radiopaque, reactive polymer that comprises a plurality of amino groups that are reactive with the multifunctional cyclic carbonate groups of the multifunctional cyclic carbonate compound. Unless indicated otherwise, as used herein the prefix “poly” means two or more. Particular multifunctional cyclic carbonate compounds and particular radiopaque, reactive multi-arm polymers are described below.

Multifunctional cyclic carbonate compounds for use herein include those that contain two or more cyclic carbonate groups, which may be selected, for example, from five-membered cyclic carbonates (e.g.,

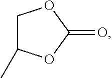

etc.), six-membered cyclic carbonates (e.g.,

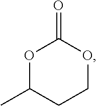

etc.), seven-membered cyclic carbonates (e.g.,

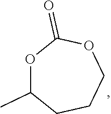

etc.), and eight-membered cyclic carbonates (e.g.,

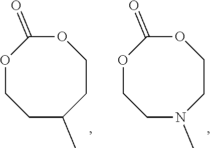

etc.), among others.

Various synthetic paths are known for the production of multifunctional cyclic carbonate compounds. For example, multifunctional cyclic carbonate compounds can be formed from compounds having multiple diol pairs, with each diol pair forming a cyclic carbonate ring. In this regard, 1,2-diols can form five-member cyclic carbonate compounds, 1,3-diols, with one intervening atom between the hydroxyl groups, can form six-member cyclic carbonate compounds, 1,4-diols, with two intervening atoms between the hydroxyl groups, can form seven-member cyclic carbonate compounds, and 1,5-diols, with three intervening atoms between the hydroxyl groups, can form eight-member cyclic carbonate compounds.

Cyclic carbonates may be formed, for example, by reaction between phosgene and diols, catalyzed or uncatalyzed condensation reactions between diols and CO₂, oxidative carbonylation of diols with CO and oxygen as oxidant, reaction between diols and an activated form of CO₂ such as urea or dimethyl carbonate, and oxidative carboxylation of compounds having two or more olefin groups. Aliphatic N-substituted functional eight-membered cyclic carbonates can be synthesized from N-substituted diethanolamines by intramolecular cyclization. Multifunctional cyclic carbonate compounds can also be formed from compounds having multiple cyclic ether groups including multiple epoxide groups. Consequently, a wide variety of multifunctional cyclic carbonate compounds are either available commercially or have been described in the literature including the following specific examples, among many others:

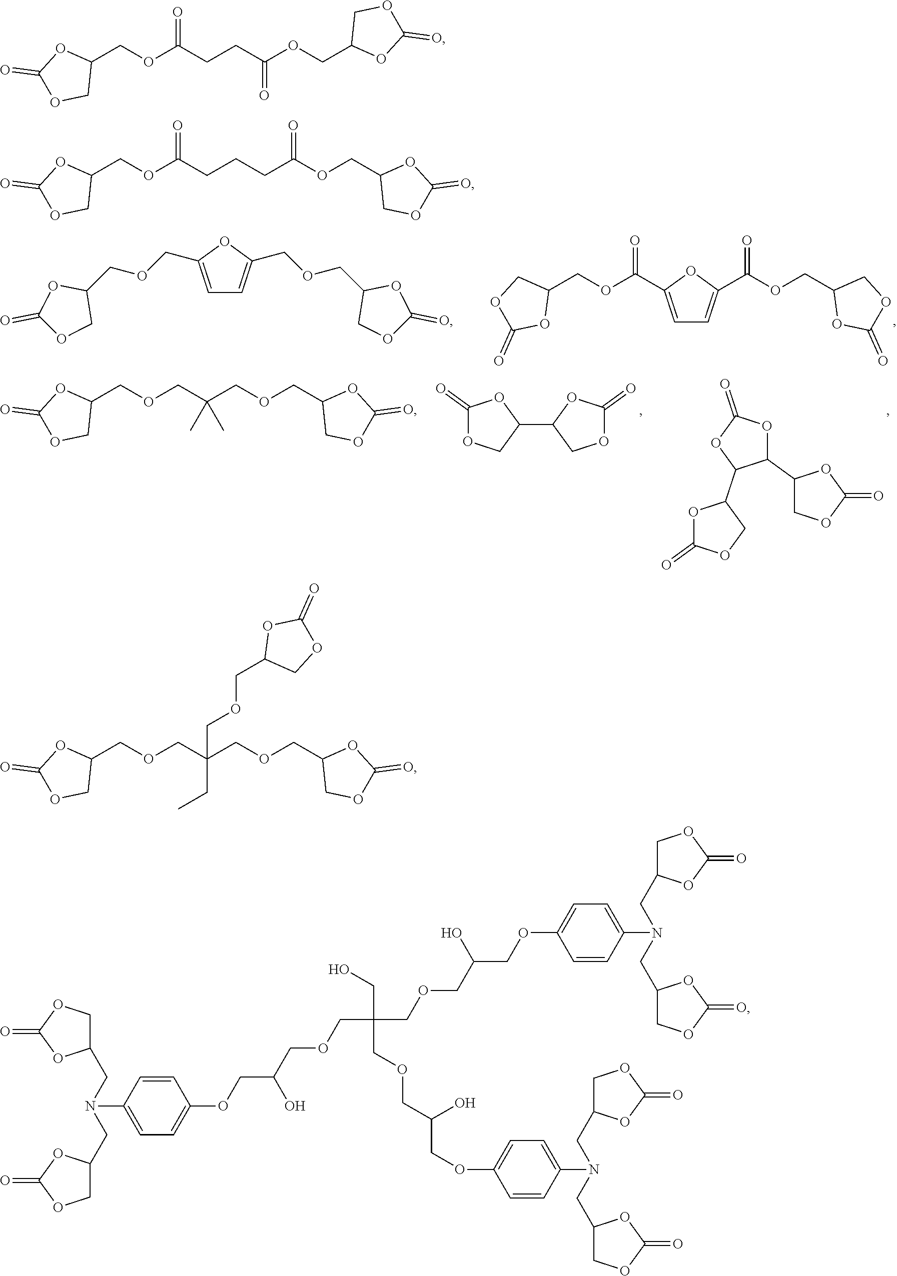

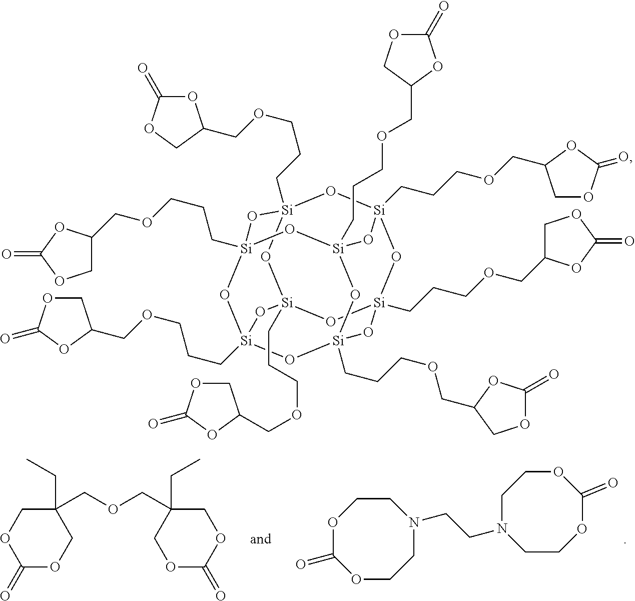

See, e.g., Aryane A. Marciniak et al., Current Opinion in Green and Sustainable Chemistry (2020), 100365, ISSN 2452-2236; Thomas M. McGuire et al., Journal of CO2 Utilization, Volume 27, October 2018, Pages 283-288; Alvaro Gomez-Lopez et al., ACS Sustainable Chem. Eng. 2021, 9, 29, 9541-9562; Michael North et al., Green Chem., 2010, 12(9), 1514-1539; Noé Fanjul-Mosteirin et al., Chemistry of Materials 2021, 33, 18, 7194-7202; Felipe de la Cruz-Martínez et al., ACS Sustainable Chem. Eng. 2019, 7, 24, 20126-20138; Özgür Capar et al., Polymer Chemistry 11(43): 6964-6970 (November 2020); Janusz Datta and Marcin Włoch, Polymer Bulletin, volume 73, pages 1459-1496 (2016); Hannes Blattmann and Rolf Mülhaupt, Macromolecules, 2016, 49, 3, 742-751; Lu Zhang et al., RSC Adv., 2017, 7, 37-46; Camille Carré et al., ChemSusChem, Volume 12, Issue 15, Aug. 8, 2019, Pages 3410-3430; and Magdalena M. Mazurek-Budzyńska et al., European Polymer Journal, Volume 84, November 2016, Pages 799-811.

Radiopaque, reactive multi-arm polymers having arms that comprise one or more end moieties having one or more radiopaque halogen groups and one or more reactive groups (e.g., amino groups) can be formed from precursor multi-arm polymers having arms that comprise one or more hydroxyl end groups or one or more amino end groups.

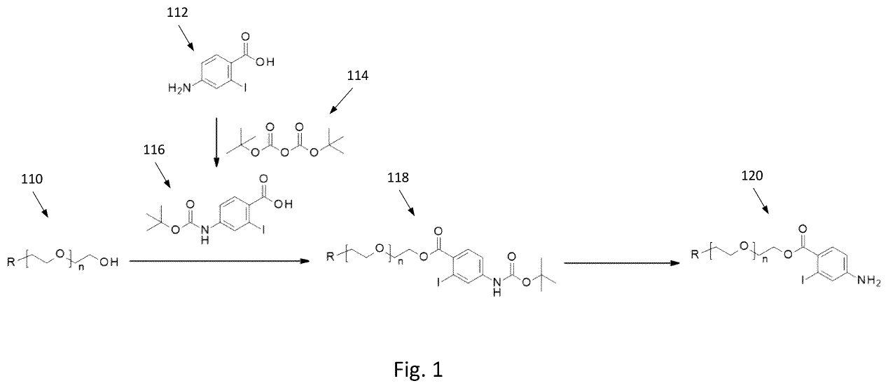

In one exemplary embodiment, a protected radiopaque precursor compound is formed by protecting the amino group(s) of a radiopaque precursor compound having one or more radiopaque halogen groups, one or more carboxy groups and one or more amino groups. In the particular embodiment shown in FIG. 1, 4-amino-2-iodobenzoic acid (112) (CAS #73655-51-3) is used as starting material to react with di-tert-butyl dicarbonate (114) (CAS #24424-99-5) to protect the amine group, thereby providing a boc-protected radiopaque compound (116).

Subsequently, hydroxyl groups of a precursor multi-arm polymer, which comprises a core region and a plurality of polymer arms (e.g., two, three, four, five, six, seven, eight, nine, ten, eleven, twelve or more polymer arms) having terminal hydroxyl groups, are reacted in an ester coupling reaction with the carboxy groups of the protected radiopaque precursor compound. This reaction step forms an intermediate multi-arm polymer, which comprises a core region and a plurality of polymer arms having end moieties that comprise one or more radiopaque halogen groups and one or more protected amino groups, where the end moieties are linked to the polymer arms by an ester linkage. In a final step, one or more amino groups of the intermediate multi-arm polymer are deprotected, for example, by exposing the intermediate multi-arm polymer to acidic conditions using an acid such as trifluoroacetic acid or hydrochloric acid, thereby providing a final radiopaque, reactive multi-arm polymer, which comprises a core region and a plurality of polymer arms having end moieties that comprise one or more radiopaque halogen groups and one or more protected amino groups, wherein the end moieties are linked to the polymers arms through an ester linkage. The ester linkage is hydrolysable in the presence of water.

More specifically, hydroxyl groups of a commercially available hydroxy-terminated 8-arm PEG (110) having a core region that comprises a polyol residue R (e.g., a pentaerythritol or a hexaglycerol residue) and eight hydroxyl-terminated polyethylene oxide arms, where n ranges from 30 to 140, may be reacted with the carboxy group of the boc-protected radiopaque compound (116) thereby forming an ester bond linkage. Such an ester-coupling reaction may be performed using a suitable coupling reagent, for instance, a carbodiimide coupling reagent such as dicyclohexylcarbodiimide (DCC) or diisopropylcarbodiimide (DIC). This reaction step forms an intermediate multi-arm polymer (118), which comprises a core region R and a plurality of polymer arms having end moieties that comprise one or more radiopaque halogen groups and one or more protected amino groups. In a further step, the boc-protected radiopaque compound (116) is deprotected to provide iodinated amino end-capped 8-Arm-PEG (120).

In an alternative embodiment, radiopaque, reactive multi-arm polymers may be formed, which comprises a core region and a plurality of polymer arms having end moieties that comprise one or more radiopaque halogen groups and one or more amino groups, wherein the end moieties are linked to the polymer arms through an amide linkage. For example, amino groups of a precursor multi-arm polymer, which comprises a core region and a plurality of polymer arms having terminal amino groups, may be reacted in an amide coupling reaction with the carboxy groups of a protected radiopaque precursor compound like that described above. This results in an intermediate multi-arm polymer, which comprises a core region and a plurality of polymer arms having end moieties that comprise one or more radiopaque halogen groups and one or more protected amino groups, where the end moieties are linked to the polymer arms by an amide linkage. The protected amino groups of the intermediate multi-arm polymer are then deprotected, for example, by exposing the intermediate multi-arm polymer to acidic conditions.

Each of the above reaction schemes require a radiopaque precursor compound having one or more radiopaque halogen groups, one or more carboxy groups and one or more amino groups. In various embodiments, this compound may comprise a monocyclic or multicyclic aromatic structure, such as a benzene, naphthalene, anthracene, phenanthrene, or tetracene structure, that is substituted with (a) one or more iodine groups, (b) one or more carboxyl groups or carboxyl-containing groups such as C₂-C₈-carboxyalkyl groups (e.g., C₂-C₅-monocarboxyalkyl groups, C₃-C₆-dicarboxyalkyl groups, C₄-C₇-tricarboxyalkyl groups, C₅-C₈-tetracarboxyalkyl groups, etc.), among others, which carboxyalkyl groups may be linked to the monocyclic or multicyclic aromatic structures directly or through any suitable linking moiety, which may be selected, for example, from amide groups, amine groups, ether groups, ester groups, or carbonate groups, among others, and (c) one or more amino groups or amino-containing groups such as C₁-C₄-aminoalkyl groups (e.g., C₁-C₄-monoaminoalkyl groups, C₁-C₄-diaminoalkyl groups, C₁-C₄-triaminoalkyl groups, C₁-C₄-tetraaminoalkyl groups, etc.), among others, which aminoalkyl groups may be linked to the monocyclic or multicyclic aromatic structures directly or through any suitable linking moiety, which may be selected, for example, from amide groups, amine groups, ether groups, ester groups, or carbonate groups, among others. Specific examples of such compounds include compounds containing one iodine group, such as the 4-amino-2-iodobenzoic acid shown in FIG. 1, compounds containing two iodine groups, such as 4-amino-2,3-diiodo-benzoic acid,

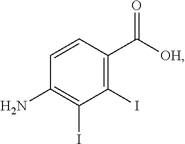 [SMILES: Nc1ccc(C(=O)O)c(I)c1I]

4-amino-2,5-diiodo-benzoic acid,

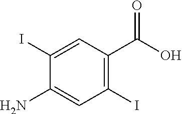 [SMILES: Nc1cc(I)c(C(=O)O)cc1I]

and 4-amino-2,6-diiodo-benzoic acid,

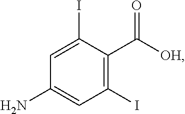 [SMILES: Nc1cc(I)c(C(=O)O)c(I)c1]

compounds containing three iodine groups, such as 4-amino-2,3,5-triiodo-benzoic acid,

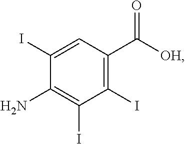 [SMILES: Nc1c(I)cc(C(=O)O)c(I)c1I]

4-amino-2,3,6-triiodo-benzoic acid,

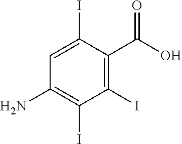 [SMILES: Nc1cc(I)c(C(=O)O)c(I)c1I]

and 4-amino-2,3,6-triiodo-benzoic acid

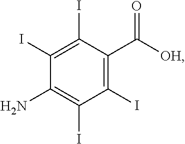 [SMILES: Nc1c(I)c(I)c(C(=O)O)c(I)c1I]

and compounds containing four iodine groups, such as 4-amino-2,3,5,6-tetraiodo-benzoic acid

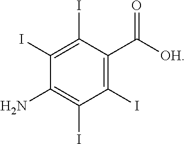 [SMILES: Nc1c(I)c(I)c(C(=O)O)c(I)c1I]

Each of the above reaction schemes also requires a precursor multi-arm polymer having a plurality of polymer arms (e.g., having two, three, four, five, six, seven, eight, nine, ten, eleven, twelve or more arms), wherein two or more polymer arms of the precursor multi-arm polymer each comprises one or more hydroxyl end groups (where ester bonds are formed) or one or more amino end groups (where amide bonds are formed).

In various embodiments, the polymer arms are hydrophilic polymer arms. Such hydrophilic polymer arms may be composed of any of a variety of synthetic, natural, or hybrid synthetic-natural polymer arms. These may be selected, for example, from the following polymer arms: polyether arms including poly(alkylene oxide) arms such as poly(ethylene oxide) (PEO) (also referred to as polyethylene glycol or PEG) arms, poly(propylene oxide) arms, poly(ethylene oxide-co-propylene oxide) arms, poly(N-vinyl pyrrolidone) arms, polyoxazoline arms including poly(2-alkyl-2-oxazoline) arms such as poly(2-methyl-2-oxazoline) arms, poly(2-ethyl-2-oxazoline) arms and poly(2-propyl-2-oxazoline) arms, poly(vinyl alcohol) arms, poly(allyl alcohol) arms, polyhydroxyethyl acrylate arms, polyhydroxyethyl methacrylate arms, PNIPAAM arms, or polysaccharide arms. Polymer arms for use in the multi-arm polymers of the present disclosure typically contain between 10 and 1000 monomer units.

In various embodiments, the polymer arms extend from a core region. In certain of these embodiments, the core region comprises a residue of a polyol comprising two or more hydroxyl groups, which is used to form the polymer arms. Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others. In certain beneficial embodiments, the core region comprises a residue of a polyol that contains two, three, four, five, six, seven, eight, nine, ten, eleven, twelve or more hydroxyl groups.

In certain of these embodiments, the core region comprises a silsesquioxane. A silsesquioxane is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, the organic groups comprise polymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers. The silicon-oxygen cores are sometimes referred to as T6, T8, T10, and T12 cage-like silicon-oxygen cores, respectively (where T=the number of tetrahedral Si vertices). In all cases each Si atom is bonded to three O atoms, which in turn connect to other Si atoms. Silsesquioxanes include compounds of the chemical formula [RSiO_{3/2}]ₙ, where n is an integer of at least 6, commonly 6, 8, 10 or 12 (thereby having T₆, T₈, T₁₀ or T₁₂ cage-like silicon-oxygen core, respectively), and where R may be selected from an array of organic functional groups such as alkyl groups, aryl groups, alkoxyl groups, and polymeric arms, among others. The T₈ cage-like silicon-oxygen cores are widely studied and have the formula [RSiO_{3/2}]₈, or equivalently R₈Si₈O₁₂. Such a structure is shown here:

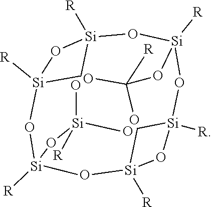

In the present disclosure, at least two R groups comprise polymer arms, and typically all R groups comprise polymer arms.

The reaction schemes described above produce radiopaque, reactive multi-arm polymers having a plurality of polymer arms (e.g., having two, three, four, five, six, seven, eight, nine, ten, eleven, twelve or more arms), wherein two or more polymer arms of the radiopaque, reactive multi-arm polymer multi-arm polymer each comprise one or more end moieties that comprise one or more radiopaque halogen groups and one or more amino groups, which end moieties are linked to the polymer arms by an ester linkage or an amide linkage.

In various embodiments, the end moieties may comprise a monocyclic or multicyclic aromatic structure, such as a benzene, naphthalene, anthracene, phenanthrene, or tetracene structure, that is substituted with (a) one or more iodine groups and (b) one or more amino groups or amino-containing groups such as C₁-C₄-aminoalkyl groups (e.g., C₁-C₄-monoaminoalkyl groups, C₁-C₄-diaminoalkyl groups, C₁-C₄-triaminoalkyl groups, C₁-C₄-tetraaminoalkyl groups, etc.), among others, which aminoalkyl groups may be linked to the monocyclic or multicyclic aromatic structures directly or through any suitable linking moiety, which may be selected, for example, from amide groups, amine groups, ether groups, ester groups, or carbonate groups, among others.

In particular embodiments, the end moieties may be selected from moieties that comprise one iodine group and an amino group, such as moieties comprising 4-amino-2-iodophenyl groups, moieties comprising two iodine groups and an amino group, such as moieties comprising 4-amino-2,3-diiodo-phenyl groups, 4-amino-2,5-diiodo-phenyl groups or 4-amino-2,6-diiodo-phenyl groups, moieties comprising three iodine groups and an amino group, such as moieties comprising 4-amino-2,3,5-triiodo-phenyl groups, 4-amino-2,3,6-triiodo-phenyl groups, or 4-amino-2,3,6-triiodo-phenyl groups, and moieties comprising four iodine groups and an amino group, such as moieties comprising 4-amino-2,3,5,6-tetraiodo-phenyl groups.

Although iodine groups are described, other radiopaque halogen groups including bromine may be employed.

As previously indicated, in some aspects, the present disclosure provides a polyurethane-based radiopaque hydrogel that comprises a crosslinked reaction product of (a) a multifunctional cyclic carbonate compound and (b) a radiopaque, reactive multi-arm polymer like that described above.

More particularly, in some aspects of the present disclosure, a polyurethane-based radiopaque hydrogel is provided that comprises a crosslinked reaction product of (a) a multifunctional cyclic carbonate compound, such as those described above, among others, and (b) a radiopaque, reactive multi-arm polymer that comprises a plurality of polymer arms that have end moieties comprising one or more radiopaque halogen groups and one or more amino groups, such as those described above, among others, which amino groups react with the multifunctional cyclic carbonate groups of the multifunctional cyclic carbonate compound to form urethane bonds.

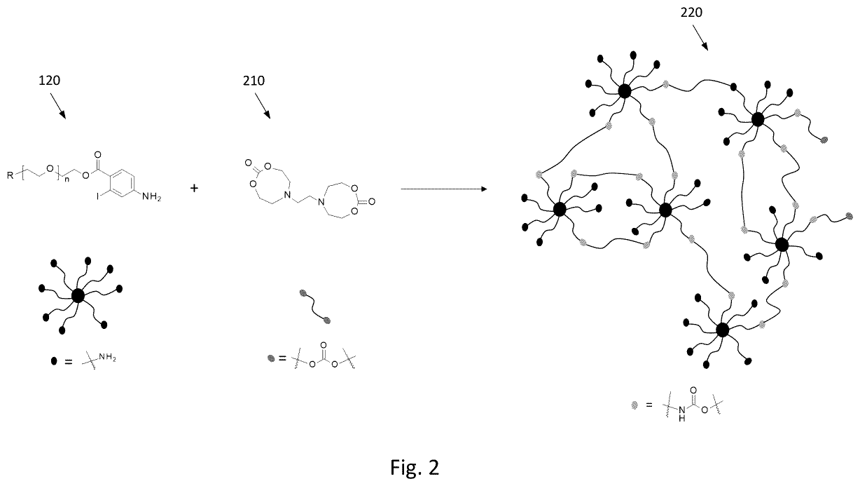

For example, as shown schematically in FIG. 2, a radiopaque, reactive multi-arm polymer like that described above, specifically a radiopaque, reactive multi-arm polymer (120) which comprises a core region R (for example, a tripentaerythritol or hexaglycerol residue core) and a plurality of hydrophilic polyethylene oxide arms (specifically, eight polyethylene oxide arms, where n ranges, for example, from 25 to 140) having end moieties that comprise a radiopaque iodine group and an amine group, is crosslinked with a multifunctional cyclic carbonate compound, specifically a bis-cyclic carbonate compound (210), which comprises two N-substituted eight-membered cyclic carbonate groups that are reactive with the amino groups of the radiopaque, reactive multi-arm polymer, by reacting the multifunctional cyclic carbonate compound with the radiopaque, reactive multi-arm polymer under basic conditions, to form a radiopaque crosslinked product (220), which may be in the form of a hydrogel when hydrated.

An advantage to this approach is that each arm of the multi-arm polymer can comprise one or more reactive amine groups and one or more reactive iodine groups, and the polyethylene oxide arms of the multi-arm polymer can be swapped out with multi-arm polymers having hydrophilic polymer arms other than polyethylene oxide arms, for example, synthetic, natural, or hybrid synthetic-natural hydrophilic polymer arms such as those described above. Another advantage of this reaction product is that a polyurethane-based crosslinked reaction product can be formed without the use of isocyanates, which have significant toxicity.

In various embodiments, the crosslinked reaction products of the present disclosure are visible under fluoroscopy. In various embodiments, such crosslinked products have a radiopacity that is greater than 100 Hounsfield units (HU), beneficially anywhere ranging from 100 HU to 250 HU to 500 HU to 750 HU to 1000 HU or more (in other words, ranging between any two of the preceding numerical values). Such crosslinked products may be formed in vivo (e.g., using a delivery device like that described below), or such crosslinked products may be formed ex vivo and subsequently administered to a subject. Such crosslinked products can be used in a wide variety of biomedical applications, including implants, medical devices, and pharmaceutical compositions.

In some aspects of the present disclosure, systems are provided that are configured to deliver (a) a multifunctional cyclic carbonate compound and (b) a radiopaque, reactive multi-arm polymer that comprises a plurality of reactive end groups that are reactive with the multifunctional cyclic carbonate groups of the multifunctional cyclic carbonate compound under conditions such that the multifunctional cyclic carbonate compound and the radiopaque, reactive multi-arm polymer crosslink with one another. In certain embodiments, those conditions comprise an environment having a basic pH, for example, a pH ranging from about 9 to about 11. Such systems can be used to form radiopaque crosslinked hydrogels, either in vivo or ex vivo.

In some aspects of the present disclosure, a system is provided that comprises (a) a first composition that comprises a multifunctional cyclic carbonate compound, for example, as described herein and (b) a second composition that comprises a radiopaque, reactive multi-arm polymer that comprises a plurality of reactive end groups that are reactive with the multifunctional cyclic carbonate groups of the multifunctional cyclic carbonate compound, for example, as described herein.

The first composition may be a first fluid composition comprising the multifunctional cyclic carbonate compound or a first dry composition that comprises the multifunctional cyclic carbonate compound, to which a suitable fluid such as water for injection, saline, etc. can be added to form a first fluid composition. In addition to the multifunctional cyclic carbonate compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the radiopaque, reactive multi-arm polymer or a second dry composition that comprises the radiopaque, reactive multi-arm polymer, to which a suitable fluid such as water for injection, saline, etc. can be added to form a second fluid composition. In addition to the radiopaque, reactive multi-arm polymer, the second composition may further comprise additional agents, including therapeutic agents, imaging agents, colorants, tonicity adjusting agents, suspension agents, wetting agents, and pH adjusting agents as described below.

In some embodiments, the multifunctional cyclic carbonate compound is initially combined with the radiopaque, reactive multi-arm polymer at an acidic pH at which crosslinking between the amino groups of the radiopaque, reactive multi-arm polymer and the cyclic carbonate groups of the multifunctional cyclic carbonate compound is suppressed. Then, when crosslinking is desired, a pH of the mixture of the multifunctional cyclic carbonate compound and the radiopaque, reactive multi-arm polymer is changed from an acidic pH to a basic pH, leading to crosslinking between same, thereby forming the crosslinked product.

In some embodiments, the system comprises (a) a first composition that comprises a multifunctional cyclic carbonate compound as described hereinabove and a radiopaque, reactive multi-arm polymer as described hereinabove, (b) a second composition, specifically, an accelerant composition, that contains an accelerant that is configured to accelerate crosslinking reaction between the multifunctional cyclic carbonate compound and the radiopaque, reactive multi-arm polymer, and (c) optionally, an acidic buffering composition in solution form or a neutral aqueous solution such as water for injection or saline.

The first composition may be a prepared fluid composition comprising the multifunctional cyclic carbonate compound and the radiopaque, reactive multi-arm polymer that is buffered to an acidic pH. The first composition may be a first dry composition that comprises the multifunctional cyclic carbonate compound and the radiopaque, reactive multi-arm polymer, to which the acidic buffering composition in solution form can be added to form a prepared fluid composition comprising the multifunctional cyclic carbonate compound and the radiopaque, reactive multi-arm polymer that is buffered to an acidic pH. The first composition may be a first dry composition that comprises the multifunctional cyclic carbonate compound, the radiopaque, reactive multi-arm polymer and an acidic buffering composition in dry form, to which a suitable aqueous fluid such as water for injection, saline, etc. can be added to form a prepared fluid composition comprising the multifunctional cyclic carbonate compound and the radiopaque, reactive multi-arm polymer that is buffered to an acidic pH. In some embodiments, for example, the acidic buffering composition, whether in solution for or in dry form, may comprise monobasic sodium phosphate, among other possibilities. The prepared fluid composition comprising the multifunctional cyclic carbonate compound and the radiopaque, reactive multi-arm polymer that is buffered to an acidic pH may have a pH ranging, for example, from about 3 to about 5. In addition to the multifunctional cyclic carbonate compound and the radiopaque, reactive multi-arm polymer, the first composition may further comprise additional agents, including therapeutic agents, imaging agents, colorants, tonicity adjusting agents, suspension agents, wetting agents, and pH adjusting agents as described below.

In a particular example, a syringe may be provided that contains a solution that is buffered to an acidic pH, and a vial may be provided that comprises a dry composition (e.g., a powder) that comprises the multifunctional cyclic carbonate compound and the radiopaque, reactive multi-arm polymer. The syringe may then be used to inject the solution that is buffered to an acidic pH into the vial containing the multifunctional cyclic carbonate compound and the radiopaque, reactive multi-arm polymer, to form a prepared fluid composition comprising the multifunctional cyclic carbonate compound and the radiopaque, reactive multi-arm polymer that is buffered to an acidic pH, which can be withdrawn back into the syringe for administration.

In another particular example, a syringe may be provided that contains an aqueous solution such as water for injection or saline, and a vial may be provided that comprises a dry composition (e.g., a powder) that comprises the multifunctional cyclic carbonate compound, the radiopaque, reactive multi-arm polymer, and an acidic buffering composition in dry form. The syringe may then be used to inject the aqueous solution into the vial containing the multifunctional cyclic carbonate compound and the radiopaque, reactive multi-arm polymer, to form a prepared fluid composition comprising the multifunctional cyclic carbonate compound and the radiopaque, reactive multi-arm polymer that is buffered to an acidic pH, which can be withdrawn back into the syringe for administration.

In other embodiments, the system comprises (a) a first composition that comprises a multifunctional cyclic carbonate compound as described hereinabove, (b) a second composition that comprises a radiopaque, reactive multi-arm polymer as described hereinabove, and (c) a third composition, specifically, an accelerant composition, that contains an accelerant that is configured to accelerate crosslinking reaction between the multifunctional cyclic carbonate compound and the radiopaque, reactive multi-arm polymer.

The first composition may be a first fluid composition comprising the multifunctional cyclic carbonate compound that is buffered to an acidic pH or a first dry composition that comprises the multifunctional cyclic carbonate compound and acidic buffering composition in dry form, to which a suitable fluid such as water for injection, saline, etc. can be added to form a first fluid composition comprising the multifunctional cyclic carbonate compound that is buffered to an acidic pH. In some embodiments, for example, the acidic buffering composition may comprise monobasic sodium phosphate, among other possibilities. The first fluid composition comprising the multifunctional cyclic carbonate compound may have a pH ranging, for example, from about 3 to about 5. In addition to the multifunctional cyclic carbonate compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the radiopaque, reactive multi-arm polymer or a second dry composition that comprises the radiopaque, reactive multi-arm polymer from which a fluid composition is formed, for example, by the addition of a suitable fluid such as water for injection, saline, or the first fluid composition comprising the multifunctional cyclic carbonate compound that is buffered to an acidic pH. In addition to the radiopaque, reactive multi-arm polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the multifunctional cyclic carbonate compound that is buffered to an acidic pH and the second composition comprises a dry composition that comprises the radiopaque, reactive multi-arm polymer. The first composition may then be mixed with the second composition to provide a prepared fluid composition that is buffered to an acidic pH and comprises the multifunctional cyclic carbonate compound and the radiopaque, reactive multi-arm polymer. In a particular example, a syringe may be provided that contains a first fluid composition comprising the multifunctional cyclic carbonate compound that is buffered to an acidic pH, and a vial may be provided that comprises a dry composition (e.g., a powder) that comprises the radiopaque, reactive multi-arm polymer. The syringe may then be used to inject the first fluid composition into the vial containing the radiopaque, reactive multi-arm polymer to form a prepared fluid composition that contains the multifunctional cyclic carbonate compound and the radiopaque, reactive multi-arm polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11. In addition to the above, the fluid accelerant composition may further comprise additional agents, including those described below.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111 In, 64Cu, 89Zr, 59Fe, 42K, 82Rb, 24Na, 45Ti, 44Sc, 51Cr and 177Lu, among others, and (f) radiocontrast agents (beyond the radiopaque iodine atoms that are present) such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block copolymers, etc.), among others, and pH adjusting agents including various buffer solutes.

A prepared fluid composition that is buffered to an acidic pH and comprises the multifunctional cyclic carbonate compound and the radiopaque, reactive multi-arm polymer as described above, and a fluid accelerant composition that is buffered to basic pH as described above, may be combined form radiopaque crosslinked hydrogels, either in vivo or ex vivo.

In various embodiments, a system is provided that include one or more delivery devices for delivering first and second compositions to a subject.

In some embodiments, the system may include a delivery device that comprises a first reservoir that contains a first composition that comprises a multifunctional cyclic carbonate compound as described above and a second reservoir that contains a second composition that comprises a radiopaque, reactive multi-arm polymer that comprises a plurality of reactive end groups that are reactive with the multifunctional cyclic carbonate groups of the multifunctional cyclic carbonate compound as described above.

In some embodiments, the system may include a delivery device that comprises a first reservoir that contains a first composition that comprises the multifunctional cyclic carbonate compound and the radiopaque, reactive multi-arm polymer and is buffered to an acidic pH, such as the prepared fluid composition previously described, and a second reservoir that contains second composition, such as the fluid accelerant composition previously described. In either case, during operation, the first composition and second composition are dispensed from the first and second reservoirs and combined, whereupon the multifunctional cyclic carbonate compound and the radiopaque, reactive multi-arm polymer and crosslink with one another to form a radiopaque crosslinked hydrogel.

Regardless of the first and second compositions selected, in particular embodiments, the system may include a delivery device that comprises a double-barrel syringe, which includes first barrel having a first barrel outlet, which first barrel contains the first composition, a first plunger that is movable in the first barrel, a second barrel having a second barrel outlet, which second barrel contains the second composition, and a second plunger that is movable in the second barrel.

In some embodiments, the device may further comprise a mixing section having a first mixing section inlet in fluid communication with the first barrel outlet, a second mixing section inlet in fluid communication with the second barrel outlet, and a mixing section outlet.

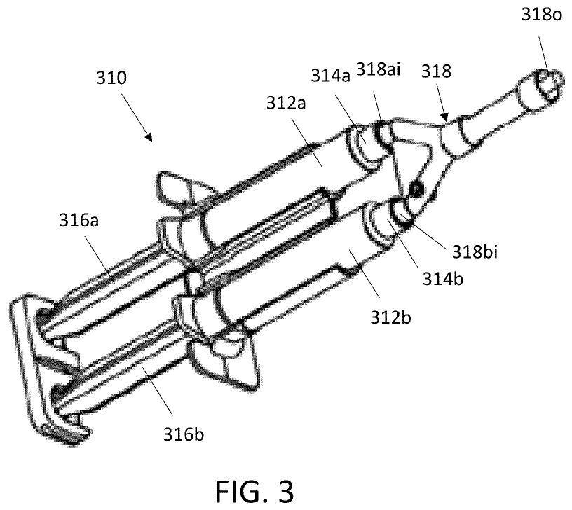

In particular embodiments, and with reference to FIG. 3, the system may include a delivery device 310 that comprises a double-barrel syringe, which includes first barrel 312a having a first barrel outlet 314a, which first barrel contains the first composition, a first plunger 316a that is movable in the first barrel 312a, a second barrel 312b having a second barrel outlet 314b, which second barrel 312b contains the second composition, and a second plunger 316b that is movable in the second barrel 312b. In some embodiments, the device 310 may further comprise a mixing section 318 having a first mixing section inlet 318ai in fluid communication with the first barrel outlet 314a, a second mixing section inlet 318bi in fluid communication with the second barrel outlet, and a mixing section outlet 3180.

In some embodiments, the device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a radiopaque crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof be injected as a scaffold, and/or the first and second fluid compositions or a fluid admixture thereof can be injected as a carrier of therapeutic agents in the treatment of diseases and cancers and the repair and regeneration of tissue, among other uses.

After administration of the compositions of the present disclosure (either separately as first and second fluid compositions that mix in vivo or as a fluid admixture of the first and second fluid compositions) a radiopaque crosslinked hydrogel is ultimately formed at the administration location.

After administration, the compositions of the present disclosure can be imaged using a suitable imaging technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant a therapeutic-agent-releasing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

As noted above, in several aspects of the present disclosure, a radiopaque crosslinked hydrogel is provided that comprises a crosslinked reaction product of (a) a multifunctional cyclic carbonate compound such as those described above and (b) a radiopaque, reactive polymer that comprises amino groups that are reactive with the multifunctional cyclic carbonate groups of the multifunctional cyclic carbonate compound.

Where formed ex vivo, radiopaque crosslinked hydrogels may be in any desired form, including a slab, a cylinder, a coating, or a particle. In some embodiments, the radiopaque crosslinked hydrogel is dried and then granulated into particles of suitable size. Granulating may be by any suitable process, for instance by grinding (including cryogrinding), crushing, milling, pounding, or the like. Sieving or other known techniques can be used to classify and fractionate the particles. Radiopaque crosslinked hydrogel particles formed using the above and other techniques may varying widely in size, for example, having an average size ranging from 50 to 950 microns.

In addition to a radiopaque crosslinked hydrogel as described above, radiopaque crosslinked hydrogel compositions in accordance with the present disclosure may contain additional agents, including therapeutic agents, imaging agents, colorants, tonicity adjusting agents, suspension agents, wetting agents, and pH adjusting agents as described above.

In various embodiments, kits are provided that include one or more delivery devices for delivering the radiopaque crosslinked hydrogel to a subject. Such systems may include one or more of the following: a syringe barrel, which may or may not contain a radiopaque crosslinked hydrogel as described herein; a vial, which may or may not contain a radiopaque crosslinked hydrogel as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the radiopaque crosslinked hydrogel may be provided in dry form (e.g., powder form) or in a form that is ready for injection, such as an injectable hydrogel form (e.g., a suspension of radiopaque crosslinked hydrogel particles).

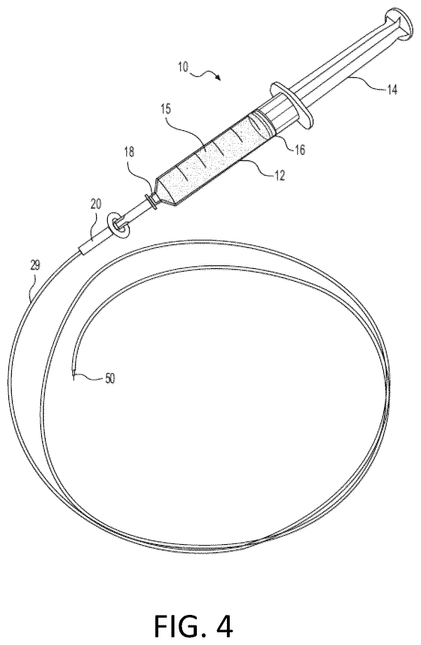

FIG. 4 illustrates a syringe 10 providing a reservoir for a radiopaque crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a radiopaque crosslinked hydrogel composition 15 for injection through the needle 50.

The radiopaque crosslinked hydrogel compositions described herein can be used for a number of purposes.

For example, radiopaque crosslinked hydrogel compositions can be injected to provide spacing between tissues, radiopaque crosslinked hydrogel compositions can be injected (e.g., in the form of blebs) to provide fiducial markers, radiopaque crosslinked hydrogel compositions can be injected for tissue augmentation or regeneration, radiopaque crosslinked hydrogel compositions can be injected as a filler or replacement for soft tissue, radiopaque crosslinked hydrogel compositions can be injected to provide mechanical support for compromised tissue, radiopaque crosslinked hydrogel compositions be injected as a scaffold, and/or radiopaque crosslinked hydrogel compositions can be injected as a carrier of therapeutic agents in the treatment of diseases and cancers and the repair and regeneration of tissue, among other uses.

After administration, the radiopaque crosslinked hydrogel compositions of the present disclosure can be imaged using a suitable imaging technique.

As seen from the above, the radiopaque crosslinked hydrogel compositions of the present disclosure may be used in a variety of medical procedures, including the following, among others: a procedure to implant a fiducial marker comprising a radiopaque crosslinked hydrogel, a procedure to implant a tissue regeneration scaffold comprising a radiopaque crosslinked hydrogel, a procedure to implant a tissue support comprising a radiopaque crosslinked hydrogel, a procedure to implant a tissue bulking agent comprising a radiopaque crosslinked hydrogel, a procedure to implant a therapeutic-agent-containing depot comprising a radiopaque crosslinked hydrogel, a tissue augmentation procedure comprising implanting a radiopaque crosslinked hydrogel, a procedure to introduce a radiopaque crosslinked hydrogel between a first tissue and a second tissue to space the first tissue from the second tissue.

The radiopaque crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Radiopaque crosslinked hydrogel compositions in accordance with the present disclosure also include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

1. A system for forming a hydrogel composition that comprises (a) a multifunctional cyclic carbonate compound and (b) a radiopaque, reactive polymer that comprises a plurality of amino groups that are reactive with the multifunctional cyclic carbonate groups of the multifunctional cyclic carbonate compound.

2. The system of claim 1, wherein the multifunctional cyclic carbonate compound comprises two or more cyclic carbonate groups selected from five-membered cyclic carbonate groups, six-membered cyclic carbonate groups, seven-membered cyclic carbonate groups and eight-membered cyclic carbonate groups.

3. The system of claim 1, wherein the radiopaque, reactive polymer comprises a plurality of hydrophilic polymer arms comprising end moieties that comprise one or more radiopaque halogen groups and one or more reactive amino groups.

4. The system of claim 3, wherein the hydrophilic polymer arms comprise one or more hydrophilic monomers selected from ethylene oxide, propylene oxide, N-vinyl pyrrolidone, oxazoline monomers, hydroxyethyl acrylate, hydroxyethyl methacrylate, PEG methyl ether acrylate or PEG methyl ether methacrylate, or PNIPAAM.

5. The system of claim 3, wherein the end moieties comprise a monocyclic or multicyclic aromatic structure that is substituted with (a) one or more iodine groups and (b) one or more amino groups or amino-containing groups.

6. The system of claim 3, wherein the end moieties are linked to the hydrophilic polymer arms through a hydrolysable ester group.

7. The system of claim 1, wherein the system comprises (a) a first composition that comprises the multifunctional cyclic carbonate compound and the radiopaque, reactive polymer and (b) an accelerant composition.

8. The system of claim 1, wherein the system comprises one or more additional agents selected from therapeutic agents, imaging agents, colorants, tonicity adjusting agents, suspension agents, wetting agents, and pH adjusting agents.

9. The system of claim 1, further comprising a delivery device.

10. A radiopaque crosslinked hydrogel composition comprising a crosslinked reaction product of (a) a multifunctional cyclic carbonate compound and (b) a radiopaque, reactive polymer that comprises a plurality of amino groups that are reactive with the multifunctional cyclic carbonate groups of the multifunctional cyclic carbonate compound.

11. The radiopaque crosslinked hydrogel composition of claim 10, wherein the multifunctional cyclic carbonate compound comprises two or more cyclic carbonate groups selected from five-membered cyclic carbonate groups, six-membered cyclic carbonate groups, seven-membered cyclic carbonate groups and eight-membered cyclic carbonate groups.

12. The radiopaque crosslinked hydrogel composition of claim 10, wherein the radiopaque, reactive polymer comprises a plurality of hydrophilic polymer arms comprising end moieties that comprise one or more radiopaque halogen groups and one or more reactive amino groups.

13. The radiopaque crosslinked hydrogel composition of claim 12, wherein the hydrophilic polymer arms comprise one or more hydrophilic monomers selected from ethylene oxide, propylene oxide, N-vinyl pyrrolidone, oxazoline monomers, hydroxyethyl acrylate, hydroxyethyl methacrylate, PEG methyl ether acrylate or PEG methyl ether methacrylate, or PNIPAAM.

14. The radiopaque crosslinked hydrogel composition of claim 12, wherein the end moieties comprise a monocyclic or multicyclic aromatic structure that is substituted with (a) one or more iodine groups and (b) one or more amino groups or amino-containing groups.

15. The radiopaque crosslinked hydrogel composition of claim 12, wherein the end moieties are linked to the hydrophilic polymer arms through a hydrolysable ester group.

16. A method of treatment comprising administering to a subject a mixture that comprises (a) a multifunctional cyclic carbonate compound and (b) a radiopaque, reactive polymer that comprises a plurality of amino groups that are reactive with the multifunctional cyclic carbonate groups of the multifunctional cyclic carbonate compound under conditions such that the multifunctional cyclic carbonate compound and the radiopaque, reactive multi-arm polymer crosslink after administration.

17. The method of claim 16, wherein the mixture comprises one or more additional agents selected from therapeutic agents, imaging agents, colorants, tonicity adjusting agents, suspension agents, wetting agents, and pH adjusting agents.

18. The method of claim 16, wherein the method comprises administering to the subject a fluid composition that has a basic pH and comprises the mixture of the multifunctional cyclic carbonate compound and the radiopaque, reactive polymer, and wherein the fluid composition crosslinks after administration in vivo to form a radiopaque crosslinked hydrogel composition.

19. The method of claim 16, wherein the method comprises administering (a) a first fluid composition that has an acidic pH and comprises the mixture of the multifunctional cyclic carbonate compound and the radiopaque, reactive polymer and (b) a second fluid composition that is buffered to a basic pH, wherein the first and second fluid compositions combine to form a fluid composition that comprises the mixture of the multifunctional cyclic carbonate compound and the radiopaque, reactive polymer and has a basic pH, and wherein the fluid composition crosslinks in vivo to form a radiopaque crosslinked hydrogel composition.

20. The method of claim 18, wherein the first fluid composition and the second fluid composition are delivered using a double barrel syringe.
